(19) United States
(12) Patent Application Publication
(10) Pub. No.: US 2026/0215918 A1
(43) Pub. Date: Jul. 30, 2026

(54) HEART VALVE IMPLANT APPARATUS

(71) Applicant: Medtronic, Inc., Minneapolis, MN (US)

(72) Inventors: Niall F. DUFFY, Galway (IE); Kevin M. MAUCH, Windsor, CA (US); Matthew E. GENOVESE, Windsor, CA (US)

(21) Appl. No.: 19/146,005

(22) PCT Filed: Jan. 17, 2024
(86) PCT No.: PCT/US2024/011751

§ 371 Date: Jul. 7, 2025

Related U.S. Application Data
(60) Provisional application No. 63/440,292, filed on Jan. 20, 2023.

(51) Int. Cl. A61F 2/24

(52) U.S. Cl. CPC A61F 2/243; A61F 2/2418; A61F 2230/0006; A61F 2250/001; A61F 2250/0036

(57) ABSTRACT

A heart valve implant apparatus includes an annular valve extending along a valve axis between an inflow end and an outflow end. An expansion apparatus includes a shaft extending coaxially with the valve axis and an engagement apparatus. The engagement apparatus includes a first base portion attached to the shaft and in a fixed position and a second base portion attached to the shaft and spaced a distance apart from the first base portion. The second base portion moves relative to the shaft. The engagement apparatus includes a first arm removably attached at one end to the valve and attached at an opposing end to the first base portion. A second arm is removably attached at one end to the valve and attached at an opposing end to the second base portion. Movement of the shaft changes the valve from a radially-contracted position to a radially-expanded position.

### CROSS-REFERENCE TO RELATED APPLICATIONS

This application claims the benefit of U.S. Provisional Application No. 63/440,292, filed Jan. 20, 2023, the entire contents of which are incorporated herein by reference.

### FIELD

The present disclosure relates generally to a heart valve implant apparatus and, more particularly, to a heart valve implant apparatus comprising an expansion apparatus for expanding a heart valve.

### BACKGROUND

It is known to provide a heart valve implant apparatus for implanting a valve within a target site of the vasculature of a patient. The heart valve can be moved from a radially-contracted position to a radially-expanded position. However, expansion of the heart valve can be difficult.

### SUMMARY

The following presents a simplified summary of the disclosure to provide a basic understanding of some aspects described in the detailed description.

In aspects, a heart valve implant apparatus comprises an annular valve extending along a valve axis between an inflow end of the valve and an outflow end of the valve. The valve comprises a lumen extending along the valve axis. An expansion apparatus is configured to engage the valve. The expansion apparatus comprises a shaft extending coaxially with the valve axis. The expansion apparatus comprises an engagement apparatus comprises a first base portion attached to the shaft and in a fixed position relative to the shaft. The engagement apparatus comprises a second base portion attached to the shaft and spaced a distance apart from the first base portion. The second base portion is configured to move relative to the shaft. The engagement apparatus comprises a first arm removably attached at one end to a first location of the valve and attached at an opposing end to the first base portion. The engagement apparatus comprises a second arm removably attached at one end to a second location of the valve and attached at an opposing end to the second base portion. A first central portion of the first arm is attached to a second central portion of the second arm. Movement of the shaft is configured to change a distance between the second base portion and the first base portion such that the valve is configured to move between a first position, in which the valve is in a radially-contracted position, and a second position, in which the valve is in a radially-expanded position.

In aspects, the second base portion is movable along a first axis, and the first arm is configured to exert a force upon the valve along a second axis, the first axis substantially perpendicular to the second axis.

In aspects, the valve comprises a first attachment portion positioned on an inner radial side of the valve and a second attachment portion positioned on the inner radial side. The first attachment portion is spaced apart from the second attachment portion along the valve axis.

In aspects, the first attachment portion is configured to removably receive the first arm and the second attachment portion is configured to removably receive the second arm. The valve comprises a locking frame member that resists movement of the valve from the radially-expanded position to the radially-contracted position.

In aspects, a heart valve implant apparatus comprises an annular valve extending along a valve axis between an inflow end of the valve and an outflow end of the valve. The valve comprises a lumen extending along the valve axis. An expansion apparatus is configured to engage the valve. The expansion apparatus comprises a shaft extending coaxially with the valve axis, with a base portion attached to the shaft. The expansion apparatus comprises a first arm extending between a first end and a second end, with the first end pivotably attached to the base portion. A second arm extends between a third end and a fourth end. The third end is pivotably attached to the second end and the fourth end is removably and pivotably attached to the valve. A central portion of the second arm pivotably attached to the shaft. Movement of the shaft relative to the base portion is configured to move the valve between a first position, in which the valve is in a radially-contracted position and an angle between the first arm and the second arm is greater than about 135 degrees, and a second position, in which the valve is in a radially-expanded position and the angle is less than about 135 degrees.

In aspects, the shaft is movable along a first axis, and the second arm is configured to exert a force upon the valve along a second axis. The first axis is substantially perpendicular to the second axis.

In aspects, the valve comprises a first attachment portion positioned at a first location on an inner radial side of the valve and a second attachment portion positioned at a second location on the inner radial side. The first attachment portion is spaced circumferentially apart from the second attachment portion about the valve axis.

In aspects, the first attachment portion is configured to removably receive the first arm and the second attachment portion is configured to removably receive the second arm.

In aspects, a distance separating the shaft from the third end and the fourth end increases as the valve moves from the first position to the second position. The valve comprises a locking frame member that resists movement of the valve from the radially-expanded position to the radially-contracted position.

In aspects, a heart valve implant apparatus comprises an annular valve extending along a valve axis between an inflow end of the valve and an outflow end of the valve. The valve comprises a lumen extending along the valve axis. An expansion apparatus is configured to engage the valve. The expansion apparatus comprises a shaft extending coaxially with the valve axis. The expansion apparatus comprises an engagement apparatus comprising a first base portion attached to the shaft and in a fixed position relative to the shaft. A second base portion is attached to the shaft and spaced a distance apart from the first base portion. The second base portion is configured to move relative to the shaft. An arm is removably attached at one end to a first location of the valve and attached at an opposing end to the second base portion. Movement of the shaft is configured to change a distance between the second base portion and the first base portion such that the valve is configured to move between a first position, in which the valve is in a radially-contracted position, and a second position, in which the valve is in a radially-expanded position.

In aspects, the second base portion is movable along a first axis, and the arm configured to exert a force upon the valve along a second axis. The first axis is substantially perpendicular to the second axis.

In aspects, the engagement apparatus further comprises a third base portion attached to the shaft and in a fixed position relative to the shaft. The engagement apparatus comprises a fourth base portion attached to the shaft and spaced a distance apart from the third base portion. The fourth base portion is configured to move relative to the shaft. A second arm is removably attached at one end to a second location of the valve and attached at an opposing end to the fourth base portion. Movement of the shaft is configured to change a second distance between the fourth base portion and the third base portion such that the valve is configured to move between the first position and the second position.

In aspects, the expansion apparatus further comprises a release apparatus configured to separate the arm from the valve.

In aspects, a heart valve implant apparatus comprises an annular valve extending along a valve axis between an inflow end of the valve and an outflow end of the valve. The valve comprises a first frame member comprising a first member portion comprising a first thickness and a second member portion comprising a second thickness that is less than the first thickness. A second frame member comprises a third member portion comprising a third thickness and a fourth member portion comprising a fourth thickness that is less than the third thickness. The first frame member is attached to the second frame member at an attachment location in which the second member portion is in contact with and attached to the fourth member portion such that a combined thickness of the second thickness and the fourth thickness at the attachment location is less than or equal to each of the first thickness and the third thickness. A locking member is attached to the first frame member and configured to contact the second frame member when the valve is in a radially-expanded position to maintain the first frame member in a fixed position relative to the second frame member.

In aspects, the locking member extends along a side of the second member portion to define an elongated opening between the locking member and the second member portion.

In aspects, the locking member contacts the third member portion when the valve is in the radially-expanded position.

In aspects, the locking member is deformable toward the second member portion.

In aspects, the valve comprises a second locking member attached to the frame member and configured to contact the second frame member when the valve is in the radially-expanded position to maintain the first frame member in the fixed position relative to the second frame member.

In aspects, the locking member and the second locking member are positioned on opposing sides of the second member portion.

In aspects, the locking member comprises a non-constant cross-sectional size along a length of the locking member.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 schematically illustrates example aspects of a heart valve in accordance with aspects of the disclosure;

FIG. 2 illustrates a side view of an expansion apparatus deploying a valve in accordance with aspects of the disclosure;

FIG. 3 illustrates a side view of an expansion apparatus deploying a valve in accordance with aspects of the disclosure;

FIG. 4 illustrates a side view of an expansion apparatus separated from a valve in accordance with aspects of the disclosure;

FIG. 5 illustrates a side view of an attachment portion of the valve in accordance with aspects of the disclosure;

FIG. 6 illustrates a side view of an expansion apparatus deploying a valve in accordance with aspects of the disclosure;

FIG. 7 illustrates a side view of an expansion apparatus deploying a valve in accordance with aspects of the disclosure;

FIG. 8 illustrates a side view of an expansion apparatus deploying a valve in accordance with aspects of the disclosure;

FIG. 9 illustrates a side view of an expansion apparatus deploying a valve in accordance with aspects of the disclosure;

FIG. 10 illustrates a side view of a portion of the valve in a radially-compressed position in accordance with aspects of the disclosure;

FIG. 11 illustrates a side view of a portion of the valve in a radially-expanded position in accordance with aspects of the disclosure;

FIG. 12 illustrates a cross-sectional view of the portion of the valve in accordance with aspects of the disclosure.

FIG. 13 illustrates an enlarged view of a portion of a frame member of the valve in accordance with aspects of the disclosure;

FIG. 14 illustrates an enlarged view of a portion of a frame member of the valve as the valve moves from the radially-compressed position to the radially-expanded position in accordance with aspects of the disclosure; and

FIG. 15 illustrates an enlarged view of a portion of a frame member of the valve in accordance with aspects of the disclosure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein—for example up, down, right, left, front, back, top, bottom, upper, lower, etc.—are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. For example, a “substantially planar” surface is intended to denote a surface that is planar or approximately planar. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

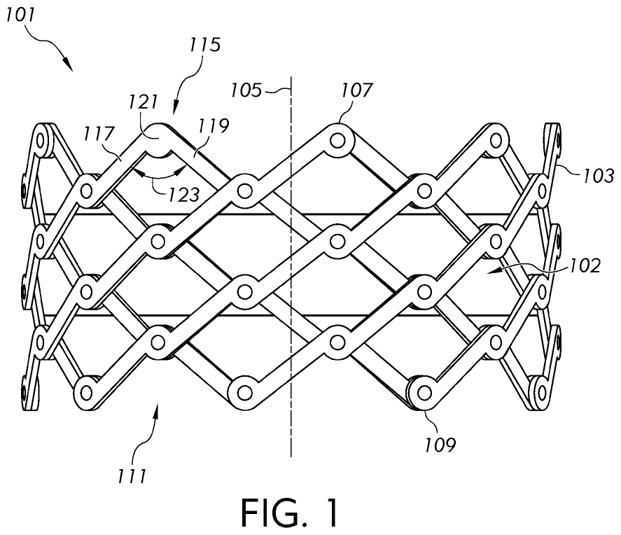

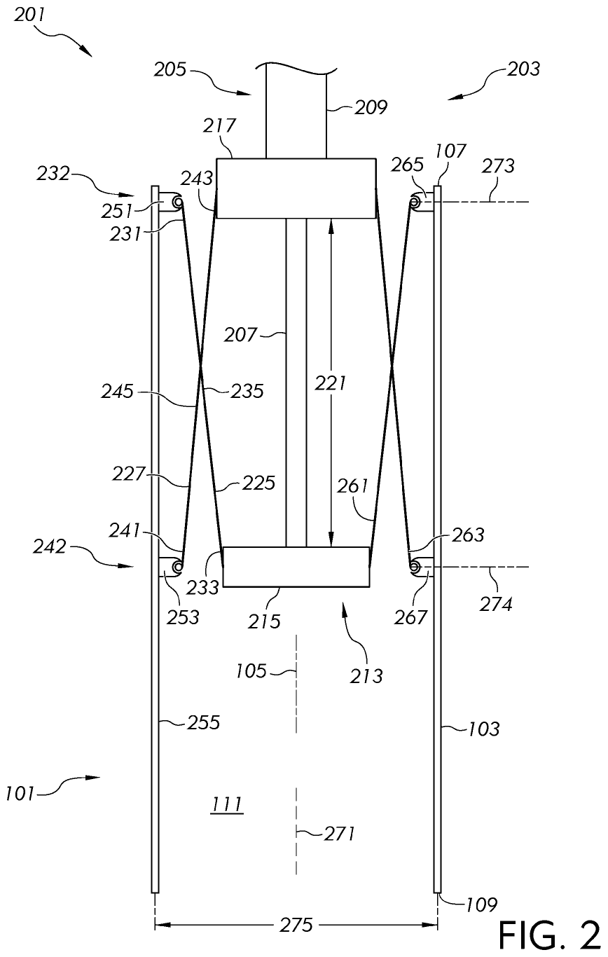

FIG. 1 illustrates an annular heart valve prosthesis 101 (e.g., hereinafter “valve 101” or “valve prosthesis 101”) for use with a heart valve implant apparatus 201 (e.g., illustrated in FIG. 2). The valve 101 can be delivered to a desired location within a body of a patient, for example, a patient's heart. The valve 101 can be moved between a radially-compressed position and a radially-expanded position, with the valve 101 in the radially-expanded position in FIG. 1. The valve 101 can move through a vasculature of the patient while in the radially-compressed position, and, upon reaching the desired location, can move to the radially-expanded position. The valve prosthesis 101 may comprise one or more structures associated with a heart valve, for example, a frame 103, valve leaflets attached to the frame 103, commissures, etc. In FIG. 1, an exemplary transcatheter aortic heart valve 102 is shown, although in other embodiments a mitral valve, tricuspid valve, or pulmonary valve may be provided without departing from the scope of the disclosure. For purposes of illustrative clarity and not limitation, the transcatheter aortic heart valve 102 is not shown in other drawing figures with the understanding that one or more features of such a heart valve 102 could be employed alone or in combination with the embodiments disclosed herein. In aspects, the valve prosthesis 101 can extend along a valve axis 105 between an inflow end 107 of the valve 101 and an outflow end 109 of the valve 101. The valve 101 can comprise a lumen 111 extending along the valve axis 105 between the inflow end 107 and the outflow end 109. The lumen 111 may be circumferentially bounded by the frame 103.

FIG. 2 illustrates the heart valve implant apparatus 201 comprising an expansion apparatus 203 and the valve 101, wherein the expansion apparatus 203 can function to mechanically expand the valve 101 from the radially-compressed position to the radially-expanded position. For purposes of illustration and to more clearly illustrate portions of the expansion apparatus, the valve 101 is illustrated schematically in FIGS. 2-15, however, in operation, the valve 101 may comprise a structure that is similar or identical to the structure illustrated in FIGS. 1 and 10-15. The expansion apparatus 203 can engage the valve 101, for example, by contacting an inner radial side of the frame 103. The expansion apparatus 203 can comprise a shaft 205 extending coaxially with the valve axis 105. The shaft 205 can extend at least partially into the lumen 111 of the valve 101. In aspects, the shaft 205 can comprise a first shaft portion 207 and a second shaft portion 209. The first shaft portion 207 and the second shaft portion 209 can extend substantially co-axially, with the first shaft portion 207 received within the second shaft portion 209. The first shaft portion 207 can extend from an end of the second shaft portion 209 and into the lumen 111, such that the first shaft portion 207 can extend a greater distance into the lumen 111 than the second shaft portion 209.

In aspects, the expansion apparatus 203 can comprise an engagement apparatus 213, wherein the engagement apparatus 213 comprises the portion of the expansion apparatus 203 that contacts the valve 101 and moves the valve 101 from the radially-compressed position to the radially-expanded position. For example, the engagement apparatus 213 can comprise a first base portion 215 and a second base portion 217. The first base portion 215 can be attached to the shaft 205, for example, by being attached to and extending circumferentially around the first shaft portion 207. The first base portion 215 can be in a fixed position relative to the shaft 205, such that the first base portion 215 may not move relative to the first shaft portion 207. In aspects, the first base portion 215 can be attached to an end of the first shaft portion 207 of the shaft 205, such that the first base portion 215 can be positioned within the lumen 111 when the valve 101 is in the radially-compressed position. The second base portion 217 can be attached to the shaft 205, for example, by being attached to and extending circumferentially around the second shaft portion 209. The second base portion 217 can be in a fixed position relative to the second shaft portion 209, such that the second base portion 217 and the second shaft portion 209 can move relative to the first shaft portion 207 and the first base portion 215. In aspects, the second base portion 217 can be attached to an end of the second shaft portion 209 of the shaft 205, for example, by being positioned at an intersection or junction of the first shaft portion 207 and the second shaft portion 209. The second base portion 217 can be spaced a first distance 221 apart from the first base portion 215 when the valve 101 is in the radially-compressed position. The second base portion 217 can move relative to the shaft 205 to alter the first distance 221 between the first base portion 215 and the second base portion 217.

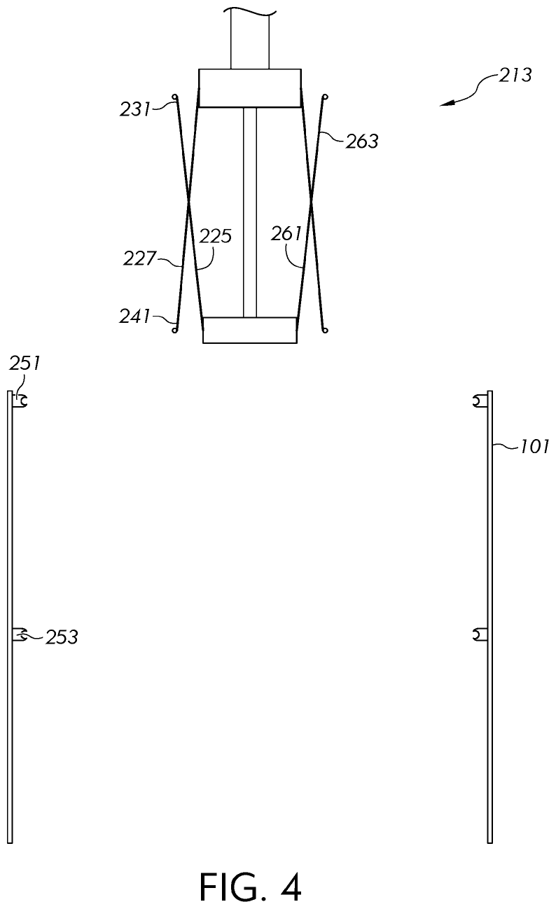

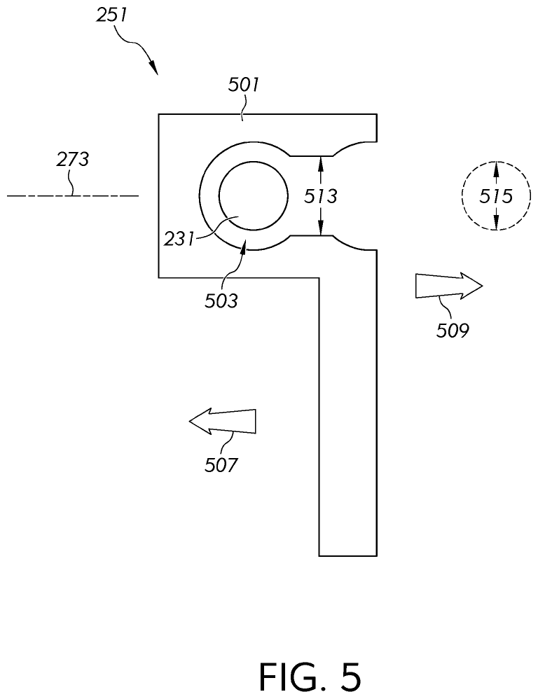

The engagement apparatus 213 can comprise one or more arms that can be attached to, and extend between, the valve 101 and the base portions 215, 217. For example, the engagement apparatus 213 can comprise a first arm 225 and a second arm 227. The first arm 225 can be removably attached at a first end 231 to a first location 232 of the valve 101 and attached at an opposing second end 233 to the first base portion 215. In aspects, the first end 231 is movable, for example, pivotable relative to the valve 101 and the second end 233 is movable, for example, pivotable relative to the first base portion 215. The first arm 225 can extend linearly between the first end 231 and the second end 233, with the first arm 225 comprising a first central portion 235 located between the first end 231 and the second end 233. In aspects, the first location 232 can comprise an axial location on the valve 101 along the valve axis 105, for example, with the first location 232 located at the inflow end 107. As will be explained relative to FIGS. 4-5, by being removably attached, the first end 231 can, at one point in time be attached to the valve 101, and, at another point in time when an operator or physician desires removal, can be detached and separated from the valve 101, such as to, for example, percutaneously deliver and implant a transcatheter heart valve prosthesis within a patient.

The second arm 227 can be removably attached at a first end 241 to a second location 242 of the valve 101 and attached at an opposing second end 243 to the second base portion 217. In aspects, the first end 241 is movable, for example, pivotable relative to the valve 101 and the second end 243 is movable, for example, pivotable relative to the second base portion 217. The second arm 227 can extend linearly between the first end 241 and the second end 243, with the second arm 227 comprising a second central portion 245 located between the first end 241 and the second end 243. In aspects, the second location 242 can comprise an axial location on the valve 101 along the valve axis 105, for example, with the second location 242 located a distance from the first location 232 and the inflow end 107, such that a distance separating the second location 242 and the outflow end 109 is less than a distance separating the first location 232 and the outflow end 109. As will be explained relative to FIGS. 4-5, by being removably attached, the first end 241 can, at one point in time be attached to the valve 101, and, at another point in time when an operator or physician desires removal, can be detached and separated from the valve 101. In aspects, the first central portion 235 of the first arm 225 can be attached to the second central portion 245 of the second arm 227.

The valve 101 can comprise one or more attachment portions, for example, a first attachment portion 251 and a second attachment portion 253. The first attachment portion 251 and the second attachment portion 253 may be positioned on an inner radial side 255 of the frame 103 of the valve 101. The first attachment portion 251 may be spaced apart from the second attachment portion 253 along the valve axis 105, for example, with the first attachment portion 251 positioned at the first location 232 and the second attachment portion 253 positioned at the second location 242. The first attachment portion 251 can removably receive the first arm 225, for example, the first end 231, such that the first arm 225 is attached to and pivotable relative to the first attachment portion 251. The second attachment portion 253 can removably receive the second arm 227, for example, the first end 241, such that the second arm 227 is attached to and pivotable relative to the second attachment portion 253.

In aspects, the engagement apparatus 213 is not limited to comprising the first arm 225, the second arm 227, the first attachment portion 251, and the second attachment portion 253. Rather, in aspects, the engagement apparatus 213 can comprise additional arms and attachment portions that can facilitate radial expansion of the valve 101. For example, the engagement apparatus 213 can comprise a third arm 261 and a fourth arm 263. The third arm 261 can be attached to the first base portion 215 at one end and removably attached to a third attachment portion 265 at an opposing end. The fourth arm 263 can be attached to the second base portion 217 at one end and removably attached to a fourth attachment portion 267 at an opposing end. In this way, the third arm 261 can be substantially identical to the first arm 225, with the third arm 261 circumferentially offset from the first arm 225 by about 180 degrees about the valve axis 105. Likewise, the fourth arm 263 can be substantially identical to the second arm 227, with the fourth arm 263 circumferentially offset from the second arm 227 by about 180 degrees about the valve axis 105. The third attachment portion 265 can be substantially identical to the first attachment portion 251 and circumferentially offset by about 180 degrees, with the third attachment portion 265 located at the first location 232. The fourth attachment portion 267 can be substantially identical to the second attachment portion 253 and circumferentially offset by about 180 degrees, with the fourth attachment portion 267 located at the second location 242. As such, the first and second arm 225, 227 can be located on one side of the shaft 205 while the third and fourth arms 261, 263 can be located on an opposing side of the shaft 205.

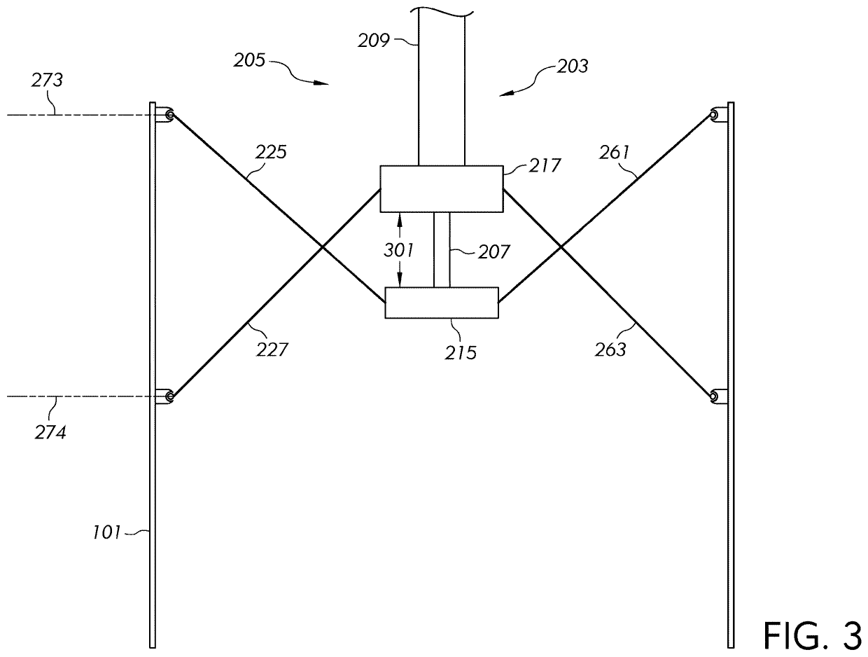

In aspects, the second base portion 217 can be movable along a first axis 271, and the first arm 225 and the second arm 227 can exert a force upon the valve 101 along a second axis 273 and a third axis 274, with the first axis 271 substantially perpendicular to the second axis 273 and the third axis 274. For example, in the initial position illustrated in FIG. 2, the expansion apparatus 203 may maintain the valve 101 in the radially-compressed position, wherein the valve 101 comprises a first diameter 275. Referring to FIG. 3, the expansion apparatus 203 can move the valve 101 from the radially-compressed position of FIG. 2 to a radially-expanded position of FIG. 3. For example, the second shaft portion 209 can move, which can cause the second base portion 217 to move as well. In aspects, the second shaft portion 209 and the second base portion 217 can move relative to the first shaft portion 207. For example, the second shaft portion 209 can move toward the valve 101, with the first shaft portion 207 telescopically received within the second shaft portion 209. As a result, the second base portion 217 can move toward the first base portion 215. In this way, movement of the shaft 205, for example, the second shaft portion 209, can change a distance between the second base portion 217 and the first base portion 215 such that the valve 101 can move between the first position of FIG. 2, in which the valve 101 is in the radially-contracted position, and the second position of FIG. 3, in which the valve 101 is in the radially-expanded position. Changing the distance can comprise, for example, reducing the distance between the second base portion 217 and the first base portion 215 from the first distance 221 (e.g., illustrated in FIG. 2) to a second distance 301.

As the second base portion 217 moves toward the first base portion 215, the arms 225, 227, 261, 263 can pivot and apply a force to the valve 101. For example, the arms 225, 227, 261, 263 can apply an outward radial force to the valve 101 in a direction parallel to the second axis 273 and third axis 274 and away from the valve axis 105. The outward radial force can cause the valve 101 to radially-expand. The second base portion 217 can continue to move toward the first base portion 215 at least until a maximum radial expansion or maximum diameter of the valve 101 is reached, at which time, the second base portion 217 and the second shaft portion 209 can stop moving. In aspects, and as related to FIGS. 2-15, the heart valve implant apparatus(es) described herein can be controlled by an operator or physician from an exterior of the patient's vasculature. The heart valve implant apparatus(es) can comprise a delivery apparatus (e.g., a sheath, a handle, etc.) that can facilitate control and movement of the expansion apparatus and valve 101. In this way, the operator or physician can control (a) movement and delivery of the expansion apparatus and valve 101 to a desired location within a patient's vasculature; (b) deployment of the valve 101 by the expansion apparatus at the desired location; and (c) retraction of the expansion apparatus from the patient's vasculature.

FIG. 4 illustrates the separation of the engagement apparatus 213 from the valve 101. For example, the first end 231 of the first arm 225 and the first end 241 of the second arm 227 can be removed from the first attachment portion 251 and the second attachment portion 253, respectively. Likewise, first ends of the third arm 261 and the fourth arm 263 can be removed from similar attachment portions of the valve 101. In this way, the engagement apparatus 213 may initially be detached from the valve 101. Upon being detached, the engagement apparatus 213 can be removed from the lumen 111 and retracted from the vasculature, thus allowing for the valve 101 to remain in the desired position.

FIG. 5 illustrates an enlarged illustration of the first attachment portion 251. It will be appreciated that the other attachment portions 253, 265, 267 may be substantially identical to the first attachment portion 251. In aspects, the first attachment portion 251 can comprise an attachment wall 501 surrounding an opening 503. The attachment wall 501 can surround the opening 503 on three sides, with a fourth side of the opening 503 being unbounded. In this way, the opening 503 can receive the first end 231 of the first arm 225 and allow for the first end 231 to be removed. For example, to insert the first end 231, the first end 231 can be aligned with the opening 503 and moved in a first movement direction 507 toward the first attachment portion 251 and along the second axis 273. FIG. 5 illustrates the first end 231 received within the opening 503, wherein the first end 231 is illustrated with solid lines. In aspects, the first end 231 can continue to move in the first movement direction 507 to apply the force to the first attachment portion 251 and cause the valve 101 to move from the radially-compressed position to the radially-expanded position.

In aspects, upon reaching the radially-expanded position (e.g., illustrated in FIG. 4), the first end 231 can be removed from the opening 503. For example, the first end 231 can be moved in a second movement direction 509 which is opposite the first movement direction 507 and away from the first attachment portion 251. In this way, the first end 231 can be retracted from the opening 503, such that the first end 231 is illustrated with dashed lines in the removed/retracted position. The opening 503 can comprise an opening size 513 that is greater than the arm size 515 of the first end 231. In this way, the first arm 225 can be inserted into and removed from the opening 503.

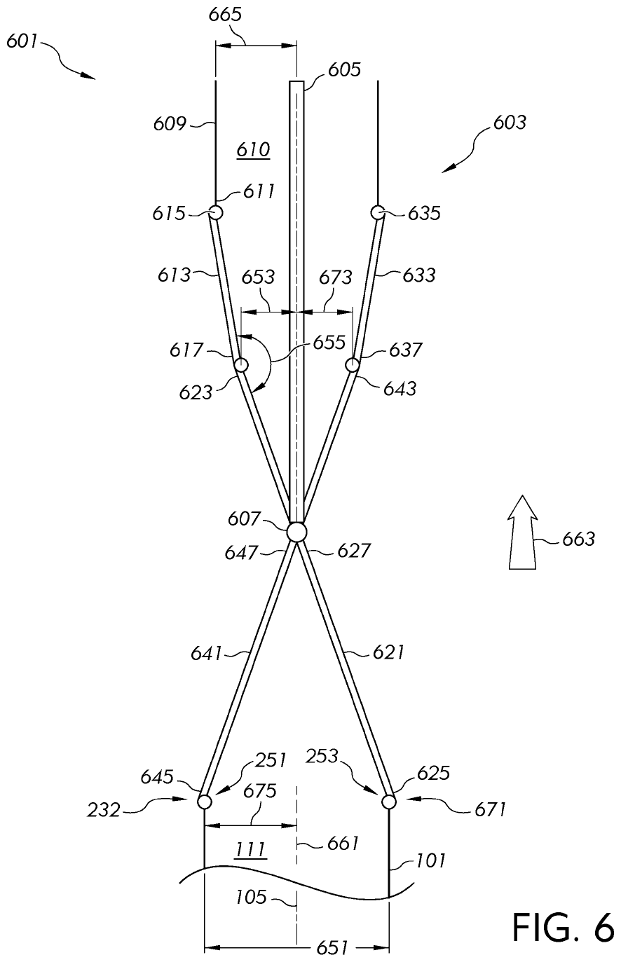

FIG. 6 illustrates additional embodiments, of a heart valve implant apparatus 601 comprising an expansion apparatus 603 and the valve 101, wherein the expansion apparatus 203 can function to mechanically expand the valve 101 from the radially-compressed position to the radially-expanded position. The expansion apparatus 603 can engage the valve 101, for example, by contacting the inner radial side of the frame 103. The expansion apparatus 603 can comprise a shaft 605 extending coaxially with the valve axis 105. In aspects, the shaft 605 may not extend into the lumen 111 such that an end 607 of the shaft 605 is located outside of the lumen 111. The expansion apparatus 603 can comprise a base portion 609 extending circumferentially around the shaft 605. The base portion 609 may be substantially hollow and define a channel 610 within which the shaft 605 extends. In aspects, the shaft 605 and the base portion 609 can extend substantially co-axially, with the end 607 of the shaft 605 extending outwardly from and not positioned within the channel 610. That is, an end 611 of the base portion 609 is located at a different axial position than the end 607 of the shaft 605.

The expansion apparatus 603 can comprise one or more arms that can be attached to, and extend between, the valve 101 and base portion 609. For example, the expansion apparatus 603 can comprise a first arm 613 that can extend between a first end 615 and a second end 617. The first end 615 can be pivotably attached to the base portion 609, for example, to the end 611 of the base portion 609. The first arm 613 can extend linearly and by being pivotable, the first arm 613 can pivot such that a distance between the shaft 605 and the second end 617 can be altered. For example, the first end 615 can remain a fixed distance from the shaft 605 due to the attachment of the first end 615 to the base portion 609. As the first arm 613 pivots relative to the base portion 609, the distance separating the second end 617 and the shaft 605 can be changed.

The expansion apparatus 603 can comprise a second arm 621 extending between a third end 623 and a fourth end 625. The third end 623 can be pivotably attached to the second end 617 of the first arm 613 and the fourth end 625 can be removably and pivotably attached to the valve 101. In aspects, the second arm 621 can comprise a central portion 627 that is pivotably attached to the shaft 605. The second arm 621 can extend linearly with the third end 643 attached to, and pivotable relative to, the second end 617 of the first arm 613. The central portion 647 of the second arm 621 can be attached to, and pivotable relative to, the end 607 of the shaft 605.

In aspects, the expansion apparatus 603 is not limited to comprising the first arm 613 and the second arm 621. Rather, in aspects, the expansion apparatus 603 can comprise additional arms that can facilitate radial expansion of the valve 101. For example, the expansion apparatus 603 can comprise a third arm 633 and a fourth arm 641. The third arm 633 can extend linearly between a first end 635 and a second end 637, with the first end 635 pivotably attached to the base portion 609. In this way, the third arm 633 may be substantially identical to the first arm 613, with the third arm 633 circumferentially offset from the first arm 613 around the base portion 609 by about 180 degrees. The fourth arm 641 can extend linearly between a third end 643 and a fourth end 645, with the third end 643 pivotably attached to the second end 637 of the third arm 633. The fourth arm 641 can comprise a central portion 647 that is pivotably attached to the shaft 605 for example, attached to, and pivotable relative to, the end 607 of the shaft 605. In this way, the fourth arm 641 may be substantially identical to the second arm 621, with the fourth arm 641 circumferentially offset from the second arm 621 by about 180 degrees.

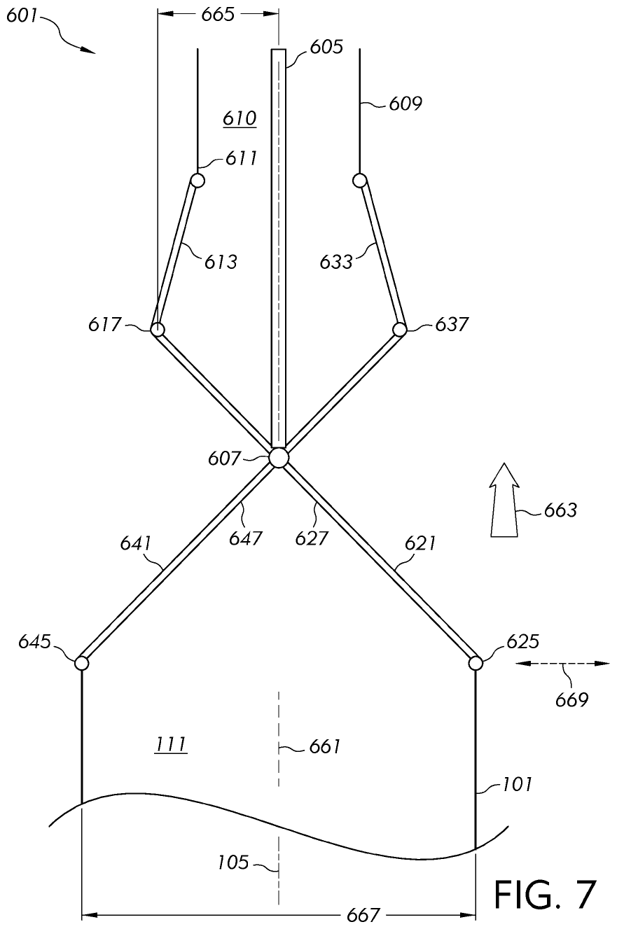

Referring to FIGS. 6-7, movement of the shaft 605 relative to the base portion 609 and/or the base portion 609 relative to the shaft 605 can cause the arms 613, 621, 633, 641 to move and exert an outward radial force on the valve 101 to move the valve 101 from the radially-contracted position (e.g., illustrated in FIG. 6) to the radially-expanded position (e.g., illustrated in FIG. 7). For example, initially in the radially-contracted position of FIG. 6, the valve 101 can comprise a first diameter 651 and the second end 617 of the first arm 613 can be spaced a first distance 653 from the shaft 605. In the radially-contracted position, an angle 655 between the first arm 613 and the second arm 621 may be greater than about 135 degrees. Referring to FIG. 7, in aspects, the shaft 605 may be movable along a first axis 661, wherein the first axis 661 extends coaxially with the valve axis 105. In aspects, the shaft 605 can be moved in a first movement direction 663 that is away from the valve 101 and toward the base portion 609, such that as the shaft 605 moves in the first movement direction 663, the shaft 605 can retract into the channel 610 of the base portion 609.

As the shaft moves in the first movement direction 663, a distance separating the end 607 of the shaft 605 from the end 611 of the base portion 609 can decrease, which can cause the second end 617 of the first arm 613 (e.g., and the second end 637 of the third arm 633) to move radially outwardly and away from the shaft 605. In this way, the second ends 617, 637 can move from the first distance 653 from the shaft 605 to a second distance 665 between the second ends 617, 637 and the shaft 605, wherein the second distance 665 is greater than the first distance 653. Due to the central portions 627, 647 being attached to the end 607 of the shaft 605, the central portions 627, 647 can be moved toward the end 611 of the base portion 609. As a result, the fourth ends 625, 645 of the second arm 621 and the fourth arm 641 can exert a force upon the valve 101 along a second axis 669, wherein the first axis 661 is substantially perpendicular to the second axis 669. In aspects, the force exerted upon the valve 101 can be in a radially-outward direction, which can cause the valve 101 to increase from the first diameter 651 to a second diameter 667, wherein the second diameter 667 is greater than the first diameter 651. In this way, movement of the shaft 605 relative to the base portion 609, for example, in the first movement direction 663, can move the valve 101 between a first position, in which the valve 101 is in a radially-contracted position and the angle 655 between the first arm 613 and the second arm 621 is greater than about 135 degrees, and a second position, in which the valve 101 is in a radially-expanded position and the angle 655 is less than about 135 degrees.

The valve 101 can comprise the first attachment portion 251 positioned at the first location 232 on the inner radial side of the valve 101, and the second attachment portion 253 at a second location 671 on the inner radial side, wherein the first attachment portion 251 is spaced circumferentially apart from the second attachment portion 253 about the valve axis 105 about 180 degrees. The first attachment portion 251 can removably receive the fourth arm 641, and the second attachment portion 253 can removably receive the second arm 621. In aspects, when the valve 101 is in the radially-contracted position illustrated in FIG. 6, the shaft 605 can be spaced a first separating distance 673 from the third end 643 of the fourth arm 641, and a second separating distance 675 from the fourth end 645 of the fourth arm 641. As the valve 101 moves from the radially-contracted position to the radially-expanded position, the first separating distance 673 and the second separating distance 675 can increase. Upon moving the valve 101 to the radially-expanded position, the expansion apparatus 603 can be detached and separated from the valve 101, for example, in a substantially identical manner as illustrated and described relative to FIGS. 4-5.

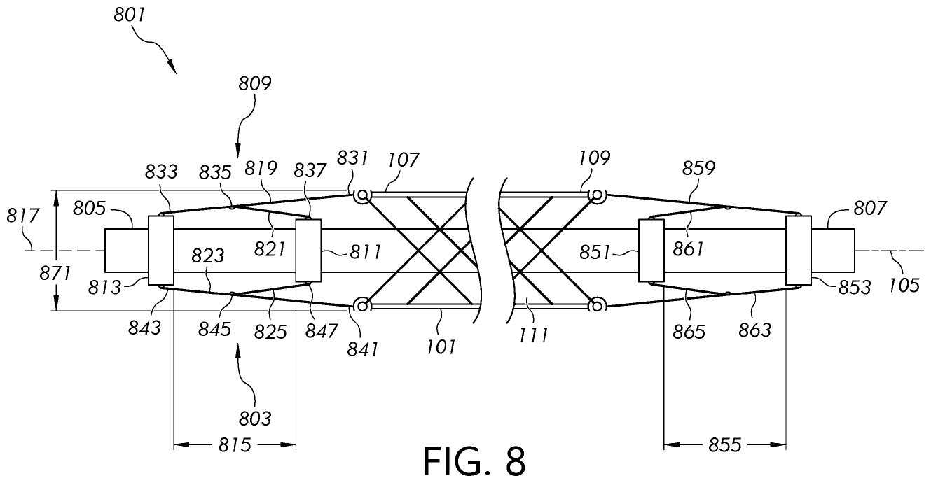

FIG. 8 illustrates additional embodiments of a heart valve implant apparatus 801 comprising an expansion apparatus 803. The expansion apparatus 803 can function to mechanically expand the valve 101 from the radially-compressed position to the radially-expanded position. The expansion apparatus 803 can comprise a shaft 805 and an engagement apparatus 809. The expansion apparatus 803 can engage the valve 101, for example, by contacting the inner radial side of the inflow end 107 and the outflow end 109 of the valve 101. The shaft 805 can extend coaxially with the valve axis 105. In aspects, the shaft 805 may extend into and through the lumen 111. In aspects, the shaft 805 can extend completely through the valve 101 such that an end 807 of the shaft 805 is located outside of the lumen 111. The expansion apparatus 803 can comprise an engagement apparatus 809 that can contact the valve 101 and move the valve 101 from the radially-compressed position to the radially-expanded position.

The engagement apparatus 809 can comprise a first base portion 811 and a second base portion 813. The first base portion 811 can extend circumferentially around the shaft 805 and may be in a fixed position relative to the shaft 805, such that the first base portion 811 may not move relative to the shaft 805. The second base portion 813 can extend circumferentially around the shaft 805 and may be spaced a first distance 815 apart from the first base portion 811 when the valve 101 is in the radially-compressed position.

In aspects, the second base portion 813 can move relative to the shaft 805. For example, rotation of the shaft 805 can cause the second base portion 813 to move axially (e.g., along a shaft axis 817 of the shaft 805) while the first base portion 811 may not move axially (e.g., remaining in the same axial position along the shaft 805). The second base portion 813 can move relative to the shaft 205 to alter the distance between the first base portion 811 and the second base portion 813. In aspects, one or more of the first base portion 811 or the second base portion 813 can be threaded, and some or all of the shaft 805 may be threaded. In this way, rotation of the shaft 805 can cause one or more of the first base portion 811 or the second base portion 813 to move axially.

In aspects, the engagement apparatus 809 can comprise one or more arms that can be attached to, and extend between, the valve 101 and the base portions 811, 813. For example, the engagement apparatus 809 can comprise a first arm 819, a second arm 821, a third arm 823, and a fourth arm 825. The first arm 819 can be removably attached at a first end 831 to a first location of the valve 101 and attached at an opposing second end 833 to the second base portion 813. The first arm 819 can extend linearly between the first end 831 and the second end 833. In aspects, the first arm 819 can be pivotable relative to the valve 101 at the first end 831 and pivotable relative to the second base portion 813 at the second end 833. In this way, the second base portion 813 can move along the shaft 805, while the first arm 819 pivots relative to the second base portion 813 and the valve 101. In aspects, the first end 831 can be attached to the inflow end 107 of the valve 101. The second arm 821 can extend linearly between a first end 835 and a second end 837. The first end 835 of the second arm 821 can be pivotably attached to the first arm 819, for example, a central portion of the first arm 819, and the second end 837 of the second arm 821 can be pivotably attached to the first base portion 811.

The third arm 823 can be removably attached at a first end 841 to a first location of the valve 101 and attached at an opposing second end 843 to the second base portion 813. The third arm 823 can extend linearly between the first end 841 and the second end 843. In aspects, the third arm 823 can be pivotable relative to the valve 101 at the first end 841 and pivotable relative to the second base portion 813 at the second end 843. In this way, the second base portion 813 can move along the shaft, while the third arm 823 pivots relative to the second base portion 813 and the valve 101. In aspects, the first end 841 can be attached to the inflow end 107 of the valve 101. The fourth arm 825 can extend linearly between a first end 845 and a second end 847. The first end 845 of the fourth arm 825 can be pivotably attached to the third arm 823, for example, a central portion of the third arm 823, and the second end 847 of the fourth arm 825 can be pivotably attached to the first base portion 811. In this way, the first arm 819 and the third arm 823 can be substantially identical and circumferentially offset by about 180 degrees about the shaft 805, and the second arm 821 and the fourth arm 825 can be substantially identical and circumferentially offset by about 180 degrees about the shaft 805.

In aspects, the engagement apparatus 809 is not limited to contacting and applying an outward radial force to the inflow end 107 of the valve 101. Rather, in aspects, the engagement apparatus 809 can comprise one or more structures for contacting and applying an outward radial force to the outflow end 109 of the valve 101. For example, the engagement apparatus 809 can comprise a third base portion 851 and a fourth base portion 853. The third base portion 851 may be substantially identical to the first base portion 811 and the fourth base portion 853 may be substantially identical to the second base portion 813, with the third base portion 851 and the fourth base portion 853 located in proximity to the outflow end 109 and spaced a distance apart from the first base portion

The third base portion 851 can extend circumferentially around the shaft 805 and may be in a fixed position relative to the shaft 805, such that the third base portion 851 may not move relative to the shaft 805. The fourth base portion 853 can extend circumferentially around the shaft 805 and may be spaced a first distance 855 apart from the third base portion 851 when the valve 101 is in the radially-compressed position. In aspects, the fourth base portion 853 can move relative to the shaft 805. For example, rotation of the shaft 805 can cause the fourth base portion 853 to move axially (e.g., along the shaft axis 817) while the third base portion 851 may be fixed and not move axially (e.g., remaining in the same axial position along the shaft 805). The fourth base portion 853 can move relative to the shaft 805 to alter the distance between the third base portion 851 and the fourth base portion 853.

In aspects, the engagement apparatus 809 can comprise one or more arms that can be attached to, and extend between, the valve 101 and the base portions 851, 853. For example, the engagement apparatus 809 can comprise a fifth arm 859, a sixth arm 861, a seventh arm 863, and an eighth arm 865. The fifth arm 859 can be substantially identical to the first arm 819, the sixth arm 861 can be substantially identical to the second arm 821, the seventh arm 863 can be substantially identical to the third arm 823, and the eighth arm 865 can be substantially identical to the fourth arm 825. In aspects, the fifth arm 859 can be removably attached at one end to a first location of the valve 101 and attached at an opposing end to the fourth base portion 853, with the fifth arm 859 extending linearly. In aspects, the fifth arm 859 can be pivotable relative to the valve 101 at the one end and pivotable relative to the fourth base portion 853 at the opposing end. In this way, the fourth base portion 853 can move along the shaft 805 while the fifth arm 859 pivots relative to the fourth base portion 853 and the valve 101. In aspects, the fifth arm 859 can be attached to the outflow end 109 of the valve 101. The sixth arm 861 can extend linearly with one end of the sixth arm 861 pivotably attached to the fifth arm 859 (e.g., a central portion of the fifth arm 859) and an opposing end of the sixth arm 861 pivotably attached to the third base portion 851.

The seventh arm 863 can be removably attached at one end to a first location of the valve 101 and attached at an opposing end to the fourth base portion 853. The seventh arm 863 can extend linearly and may be pivotable relative to the valve 101 at one end and pivotable relative to the fourth base portion 853 at the opposing end. In this way, the fourth base portion 853 can move along the shaft 805, while the seventh arm 863 can pivot relative to the fourth base portion 853 and the valve 101 while being attached to the outflow end 109 of the valve 101. In aspects, the eighth arm 865 can extend linearly and may be pivotably attached to the seventh arm 863, for example, a central portion of the seventh arm 863, while an opposing end of the eighth arm 865 can be pivotably attached to the third base portion 851. As such, the fifth arm 859 and the seventh arm 863 can be substantially identical and circumferentially offset by about 180 degrees about the shaft 805, and the sixth arm 861 and the eighth arm 865 can be substantially identical and circumferentially offset by about 180 degrees about the shaft 805.

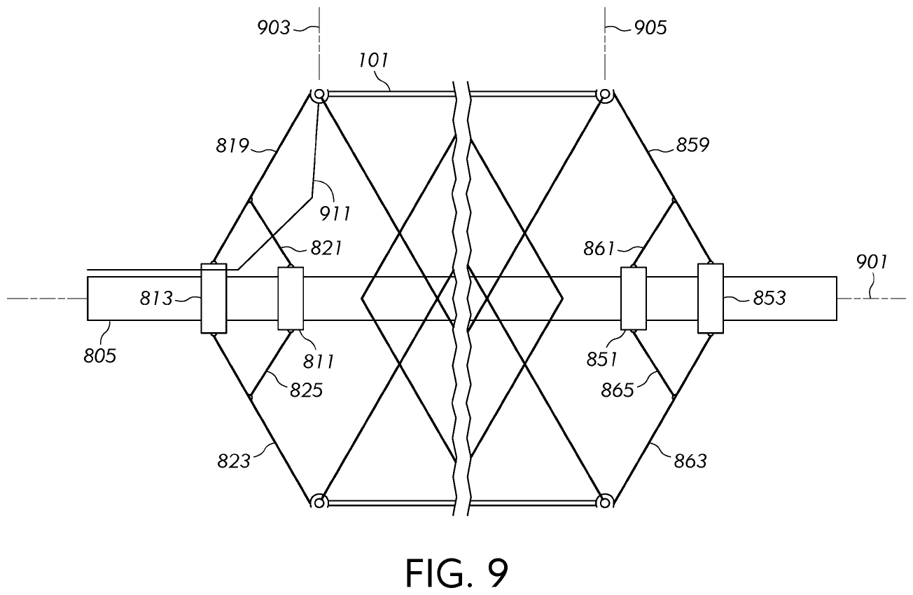

Referring to FIGS. 8-9, rotation of the shaft 805 can cause the second base portion 813 and the fourth base portion 853 to move relative to the shaft 805 and can cause the arms to move and exert an outward radial force on the valve 101 to move the valve 101 from the radially-contracted position (e.g., illustrated in FIG. 8) to the radially-expanded position (e.g., illustrated in FIG. 9). For example, initially in the radially-contracted position of FIG. 8, the valve 101 can comprise a first diameter 871 and the first base portion 811 can be spaced the first distance 815 from the second base portion 813. Rotation of the shaft 805 can cause the second base portion 813 to move toward the first base portion 811 and reduce the first distance 815. Likewise, initially in the radially-contracted position, the third base portion 851 can be spaced the first distance 855 from the fourth base portion 853. Rotation of the shaft 805 can cause the fourth base portion 853 to move toward the third base portion 851 and reduce the first distance 855.

As the second base portion 813 moves toward the first base portion 811, the first arm 819 and the third arm 823 can pivot relative to the second base portion 813, such that the first end 831 of the first arm 819 and the first end 841 of the third arm 823 can apply an outward radial force to the inflow end 107 of the valve 101. Further, the second arm 821 and the fourth arm 825 can apply a force to the first arm 819 and the third arm 823, to apply a force to the first arm 819 and the third arm 823 to facilitate outward radial expansion. Likewise, as the fourth base portion 853 moves toward the third base portion 851, the fifth arm 859 and the seventh arm 863 can pivot relative to the fourth base portion 853, such that the ends of the fifth arm 859 and the seventh arm 863 can apply an outward radial force to the outflow end 109 of the valve 101. Further, the sixth arm 861 and the eighth arm 865 can apply a force to the fifth arm 859 and the seventh arm 863, to apply a force to the fifth arm 859 and the seventh arm 863 to facilitate outward radial expansion.

Referring to FIG. 9, the second base portion 813 is movable along a first axis 901, and the arms 819, 823, 859, 863 can exert a force upon the valve 101 along a different axis (e.g., a second axis 903 and a third axis 905), with the first axis 901 substantially perpendicular to the second axis 903 and the third axis 905. As such, movement of the shaft 805 can change the distance between the second base portion 813 and the first base portion 811 such that the valve 101 can move between a first position, in which the valve 101 is in the radially-contracted position, and a second position, in which the valve 101 is in a radially-expanded position. Likewise, movement of the shaft 805 can change a second distance between the fourth base portion 853 and the third base portion 851 such that the valve 101 can move between the first position and the second position.

In aspects, the expansion apparatus 803 can comprise a release apparatus 911 that can separate the arms from the valve 101. For example, the release apparatus 911 can comprise a wire, a cable, or other apparatus. The release apparatus 911 can be attached to the first arm 819. Once the valve 101 has been moved to the radially-expanded position, the release apparatus 911 can apply a pulling force to the first arm 819 in a direction toward the shaft 805. In this way, the first arm 819 can be moved by the release apparatus 911 toward the shaft 805, thus causing the first arm 819 to detach and separate from the valve 101. Likewise, additional release apparatuses can be attached to the other arms 823, 859, 863 that are attached to the valve 101, with the additional release apparatuses configured to apply the pulling force to the other arms 823, 859, 863. In this way, the arms of the expansion apparatus 803 can be selectively detached from the valve 101 after radially-expanding the valve 101.

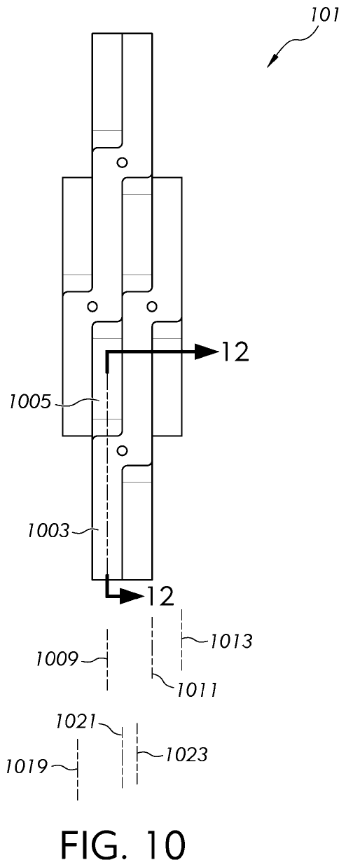

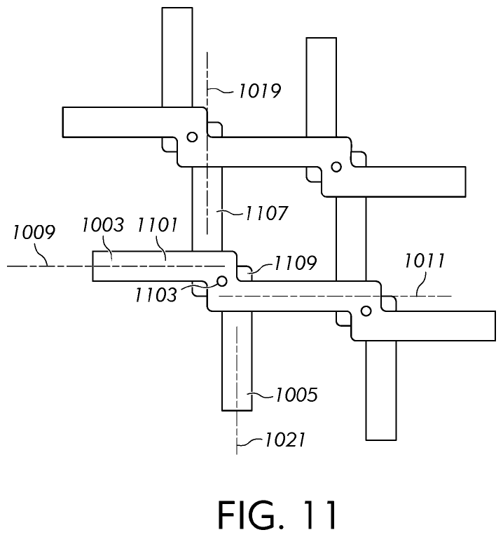

FIG. 10 illustrates a portion of the valve 101, wherein the valve 101 can be deployed with any of the previously described expansion apparatuses. As illustrated in FIG. 10, the valve 101 is in the radially-contracted position and in FIG. 11, the valve 101 in the radially-expanded position. While FIGS. 10-11 illustrate a portion of the valve 101, the remaining, un-illustrated portions of the valve 101 can be substantially identical in structure to the portion illustrated in FIGS. 10-11.

Referring to FIG. 10, the valve 101 can comprise one or more frame members, for example, a first frame member 1003, a second frame member 1005, etc. The frame members 1003, 1005, etc. may each be substantially identical. The first frame member 1003 can extend non-linearly, for example, by extending along a plurality of axes, such as, for example, a first axis 1009, a second axis 1011, a third axis 1013, etc. The axes 1009, 1011, 1013 may be substantially parallel and laterally offset. Similarly, the second frame member 1005 can extend non-linearly, for example, by extending along a plurality of axes, such as, for example, a first axis 1019, a second axis 1021, a third axis 1023, etc. The axes 1019, 1021, 1023 may be substantially parallel and laterally offset. In aspects, the first frame member 1003 can be pivotably attached to the second frame member 1005.

FIG. 11 illustrates the valve 101 in the radially-expanded position. For example, the first frame member 1003 can be rotated relative to the second frame member 1005, for example, about 90 degrees, to separate the frame members 1003, 1005. The first frame member 1003 can comprise a plurality of member portions, for example, a first member portion 1101 and a second member portion 1103. The first member portion 1101 can extend along the first axis 1009 while the second member portion 1103 can form a transition section transitioning between the first axis 1009 and the second axis 1011. The second frame member 1005 can comprise a plurality of member portions, for example, a third member portion 1107 and a fourth member portion 1109. The third member portion 1107 can extend along the first axis 1019 while the fourth member portion 1109 can form a transition section transitioning between the first axis 1019 and the second axis 1021.

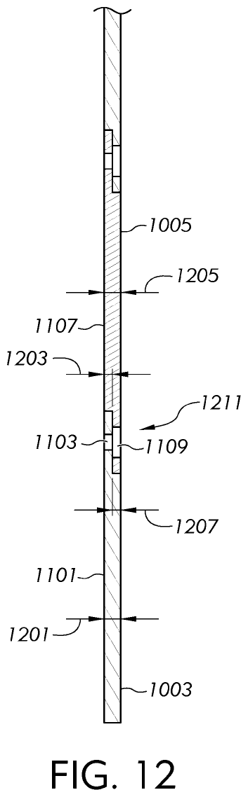

FIG. 12 illustrates a cross-sectional illustration of the first frame member 1003 and the second frame member 1005 as viewed along lines 12-12 of FIG. 10. In aspects, the first frame member 1003 can comprise the first member portion 1101 comprising a first thickness 1201 and the second member portion 1103 can comprise a second thickness 1203. The second thickness 1203 may be less than the first thickness 1201. For example, the first thickness 1201 may be about twice as thick as the second thickness 1203. As such, in this way, the first frame member 1003 can comprise a non-constant thickness along the length of the first frame member 1003. The second frame member 1005 can comprise the third member portion 1107 comprising a third thickness 1205 and the fourth member portion 1109 comprising a fourth thickness 1207. In aspects, the fourth thickness 1207 may be less than the third thickness 1205. For example, the third thickness 1205 may be about twice as thick as the fourth thickness 1207. As such, in this way, the second frame member 1005 can comprise a non-constant thickness along the length of the second frame member 1005. The first thickness 1201 and the third thickness 1205 may be substantially equal, and the second thickness 1203 and the fourth thickness 1207 may be substantially equal.

In aspects, the first frame member 1003 and the second frame member 1005 can be pivotably attached. For example, the first frame member 1003 can be attached to the second frame member 1005 at an attachment location 1211 in which the second member portion 1103 is in contact with and attached to the fourth member portion 1109. In this way, a combined thickness of the second thickness 1203 and the fourth thickness 1207 at the attachment location 1211 is less than or equal to each of the first thickness 1201 and the third thickness 1205. For example, the second member portion 1103 and the fourth member portion 1109 may be in contact and parallel to each other, such that the first frame member 1003 can pivot relative to the second frame member 1005 at the attachment location 1211 where the second member portion 1103 and the fourth member portion 1109 are in contact and attached. The combined thickness of the second member portion 1103 and the fourth member portion 1109 (e.g., the second thickness 1203 plus the fourth thickness 1207) may be less than or equal to the first thickness 1201. Likewise, the combined thickness of the second member portion 1103 and the fourth member portion 1109 (e.g., the second thickness 1203 plus the fourth thickness 1207) may be less than or equal to the third thickness 1205.

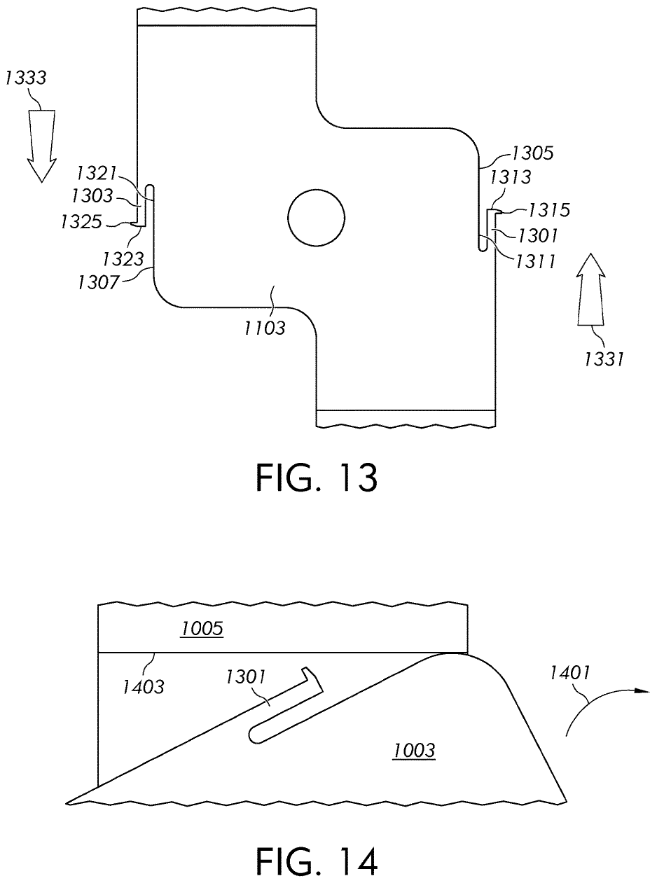

FIG. 13 illustrates an enlarged view of the second member portion 1103. In aspects, the second member portion 1103 can comprise one or more locking members that can facilitate maintaining the first frame member 1003 and the second frame member 1005 in a fixed position when the valve 101 is in the radially-expanded position. For example, the second member portion 1103 can comprise a first locking member 1301 and a second locking member 1303. The first locking member 1301 can be positioned along a first side 1305 of the second member portion 1103 and the second locking member 1303 can be positioned along an opposing second side 1307 of the second member portion 1103. In this way, the first locking member 1301 and the second member portion 1103 can be positioned on opposite sides of the second member portion 1103.

The first locking member 1301 can extend substantially parallel to a side surface on the first side 1305 of the second member portion 1103. For example, the first locking member 1301 can be spaced apart from the side surface to define a first elongated opening 1311. In this way, the first locking member 1301 can extend along the first side 1305 of the second member portion 1103 to define the first elongated opening 1311 between the first locking member 1301 and the second member portion 1103. In aspects, the first locking member 1301 can comprise a first end 1313 that is unattached to and spaced apart from the second member portion 1103. In this way, the first end 1313 can define and border an end of the first elongated opening 1311. By being unattached to and spaced apart from the second member portion 1103, the first end 1313 can allow for flexibility of the first locking member 1301, such that the first locking member 1301 can flex and move toward the second member portion 1103. In aspects, the first locking member 1301 can comprise a first locking tab 1315 located at the first end 1313. The first locking tab 1315 can define a portion of the first locking member 1301 comprising an increased thickness or width, such that the first locking member 1301 comprises a non-constant cross-sectional size along a length of the first locking member 1301. In this way, the first locking tab 1315 can project form the first locking member 1301 in a direction away from the second member portion 1103.

The second locking member 1303 may be substantially identical to the first locking member 1301. For example, the second locking member 1303 can extend substantially parallel to a side surface on the second side 1307 of the second member portion 1103. The second locking member 1303 can be spaced apart from the side surface to define a second elongated opening 1321. In this way, the second locking member 1303 can extend along the second side 1307 of the second member portion 1103 to define the second elongated opening 1321 between the second locking member 1303 and the second member portion 1103. In aspects, the second locking member 1303 can comprise a first end 1323 that is unattached to and spaced apart from the second member portion 1103. In this way, the first end 1323 can define and border an end of the second elongated opening 1321. By being unattached to and spaced apart from the second member portion 1103, the first end 1323 can allow for flexibility of the second locking member 1303, such that the second locking member 1303 can flex and move toward the second member portion 1103. In aspects, the second locking member 1303 can comprise a second locking tab 1325 located at the first end 1323. The second locking tab 1325 can define a portion of the second locking member 1303 comprising an increased thickness or width, such that the second locking member 1303 comprises a non-constant cross-sectional size along a length of the second locking member 1303. In this way, the second locking tab 1325 can project form the second locking member 1303 in a direction away from the second member portion 1103. The first locking member 1301 can extend along an axis in a first direction 1331 toward the first end 1313, and the second locking member 1303 can extend along an axis in a second direction 1333 toward the first end 1323. In aspects, the first direction 1331 may be opposite the second direction 1333 such that the first locking member 1301 can be oriented in an opposite direction from the second locking member 1303.

FIG. 14 illustrates the movement (e.g., pivoting) of the first frame member 1003 relative to the second frame member 1005 from the radially-compressed position (e.g., illustrated in FIG. 10) to the radially-expanded position (e.g., illustrated in FIG. 11). For example, the first frame member 1003 can pivot relative to the second frame member 1005 in a first rotational direction 1401, wherein the first rotational direction 1401 may be clockwise in FIG. 14. The second frame member 1005 can comprise a frame wall 1403. The frame wall 1403 can define the portion of the second frame member 1005 at the junction of the third member portion 1107 and the fourth member portion 1109. For example, the fourth member portion 1109 comprises the fourth thickness 1207 that is less than the third thickness 1205 of the third member portion 1107. The frame wall 1403 can extend between the third member portion 1107 and the fourth member portion 1109 to form an outcropping, a protuberance, projection, etc. As the first member portion 1101 pivots relative to the second member portion 1103, in aspects, the first locking member 1301 may be deformable toward the second member portion 1103. For example, the first locking tab 1315 at the first end 1313 can contact the frame wall 1403 as the first member portion 1101 pivots. The first locking tab 1315 can deflect and flex toward the second member portion 1103, causing the second elongated opening 1321 to reduce in size at the first end 1313. The second locking member 1303 can move and deform in a similar manner as the first locking member 1301.

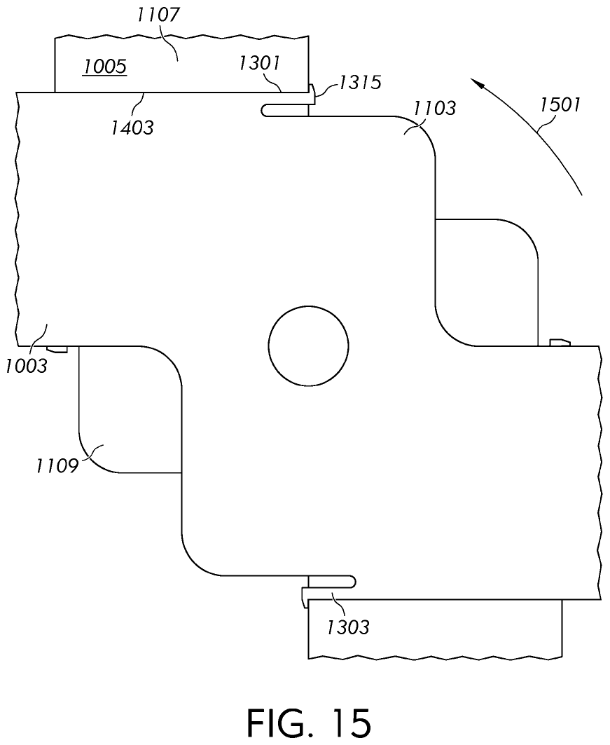

FIG. 15 illustrates an enlarged view of the second member portion 1103 when the valve 101 is in the radially-expanded position (e.g., illustrated in FIG. 11). For example, the first frame member 1003 can continue to rotate until reaching an expanded position, as illustrated. In the expanded position, the first locking member 1301 and the second locking member 1303 can engage portions of the second frame member 1005. For example, the first locking tab 1315 of the first locking member 1301 can engage and contact the third member portion 1107 of the second frame member 1005. As a result, the first locking tab 1315 can limit the first frame member 1003 from rotating relative to the second frame member 1005 in a second rotational direction 1501, which is opposite the first rotational direction 1401. The first locking member 1301, which is attached to the first frame member 1003, can contact the second frame member 1005 (e.g., the frame wall 1403 at the intersection of the third member portion 1107 and the fourth member portion 1109) when the valve 101 is in the radially-expanded position to maintain the first frame member 1003 in a fixed position relative to the second frame member 1005. The second locking member 1303 can engage a portion of the second frame member 1005 in a substantially identical manner as the first locking member 1301. As such, the locking members 1301, 1303 can assist in maintaining the valve 101 in the radially-expanded position.

Referring to FIG. 1, in aspects, the frame 103 can comprise one or more locking frame members 115. The locking frame members 115 can be used with any valves 101 illustrated and described relative to FIGS. 1-15. For example, the locking frame member 115 can comprise a material that may be work-hardened (e.g., or strain-hardened or strength-hardened), which is the strengthening of the locking frame member 115 by plastic deformation, and can occur due to the dislocation of movement within a crystal structure of the material. In aspects, the locking frame member 115 can comprise an MP35 alloy, which is a nonmagnetic material comprising cobalt, nickel, chromium, and molybdenum. In aspects, when the locking frame member 115 comprises an MP35 alloy, the locking frame member 115 can comprise, by weight, about 35% cobalt, about 35% nickel, about 20% chromium, and about 10% molybdenum. Alternatively, the locking frame member 115 can comprise an annealed stainless steel or other softer materials that can be elastically deformed and are biocompatible.

The locking frame member 115 can comprise a first member segment 117, a second member segment 119, and a junction 121 at which the first member segment 117 is attached to the second member segment 119. The locking frame member 115 can comprise a one-piece formed composite material such that the first member segment 117, the second member segment 119, and the junction 121 are a continuous, composite material. In aspects, the first member segment 117 and the second member segment 119 can flex or bend relative to one another at the junction 121. While the frame 103 is illustrated as comprising one locking frame member 115, in aspects, the frame 103 can comprise any number of locking frame members (e.g., zero or more) that may be substantially identical to the locking frame member 115. When the frame 103 comprises a plurality of locking frame members, the locking frame members may be dispersed throughout the frame. In aspects, the locking frame members may be located at ends 107, 109 of the frame 103, at axial locations between the ends 107, 109 of the frame 103, and/or in attachment with the expansion apparatuses disclosed herein. Accordingly, FIG. 1 is not intended to limit the frame 103 to the single locking frame member 115 positioned at the end.

Initially, the valve 101 may be in the radially-contracted position, such that the first member segment 117 and the second member segment 119 can be pivoted to be in closer proximity to one another, such that an angle 123 between the first member segment 117 and the second member segment 119 is less than the angle 123 illustrated in FIG. 1 (e.g., when the valve 101 and, thus, the locking frame member 115 are in the radially-expanded position). In this way, in the radially-contracted position, the first member segment 117 and the second member segment 119 may be closer to being parallel to one another. As the valve 101 moves from the radially-contracted position to the radially-expanded position (e.g., due to the expansion apparatuses illustrated in FIGS. 2-9), the locking frame member 115 can expand, which can cause the angle 123 between the first member segment 117 and the second member segment 119 to increase. As the locking frame member 115 expands, the locking frame member 115 may undergo plastic deformation (e.g., a permanent deformation or distortion occurring as a result of the force), which can cause the locking frame member 115 to work strengthen. In this way, upon reaching the radially-expanded position (e.g., illustrated in FIG. 1), the locking frame member 115 may resist moving back to the radially-contracted position and, thus, closing. Instead, the locking frame member 115 may retain the shape illustrated in FIG. 1 with the angle 123 between the first member segment 117 and the second member segment 119. A benefit of the locking frame member 115 is that the locking frame member 115 can assist in retaining the valve 101 in the radially-expanded position once the expansion apparatus moves the valve 101 from the radially-contracted position to the radially-expanded position (e.g., illustrated in FIGS. 2-15). The strength of the valve 101 and the ability of the valve 101 to resist radially-contracting from the radially-expanded state can be based, in part, on the number of locking frame member 115 provided in the frame 103. For example, with a greater number of locking frame members, the valve 101 may exhibit increased resistance to moving to the radially-contracted position as compared to one locking frame member 115 or zero locking frame members. Accordingly, in aspects, the valve 101 can comprise the locking frame member 115 that can resist movement of the valve 101 from the radially-expanded position to the radially-contracted position. In contract, at least some of the other frame members (e.g., the non-locking frame members) may be attached at a junction or node, for example, by a mechanical fastener. The non-locking frame members may not exhibit the ability to resist movement of the valve 101 from the radially-expanded position to the radially-contracted position, but, rather, may freely pivot and/or rotate at the junction or node.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. A heart valve implant apparatus comprising:
an annular valve extending along a valve axis between an inflow end of the valve and an outflow end of the valve, the valve comprising a lumen extending along the valve axis; and
an expansion apparatus configured to engage the valve, the expansion apparatus comprising:
a shaft extending coaxially with the valve axis;
an engagement apparatus comprising:
a first base portion attached to the shaft and in a fixed position relative to the shaft;
a second base portion attached to the shaft and spaced a distance apart from the first base portion, the second base portion configured to move relative to the shaft;
a first arm removably attached at one end to a first location of the valve and attached at an opposing end to the first base portion; and
a second arm removably attached at one end to a second location of the valve and attached at an opposing end to the second base portion, a first central portion of the first arm attached to a second central portion of the second arm, wherein movement of the shaft is configured to change a distance between the second base portion and the first base portion such that the valve is configured to move between a first position, in which the valve is in a radially-contracted position, and a second position, in which the valve is in a radially-expanded position.

2. The heart valve implant apparatus of claim 1, wherein the second base portion is movable along a first axis, and the first arm is configured to exert a force upon the valve along a second axis, the first axis substantially perpendicular to the second axis.

3. The heart valve implant apparatus of claim 1, wherein the valve comprises a first attachment portion positioned on an inner radial side of the valve and a second attachment portion positioned on the inner radial side, the first attachment portion spaced apart from the second attachment portion along the valve axis.

4. The heart valve implant apparatus of claim 3, wherein the first attachment portion is configured to removably receive the first arm and the second attachment portion is configured to removably receive the second arm, and wherein the valve comprises a locking frame member that resists movement of the valve from the radially-expanded position to the radially-contracted position.

5. A heart valve implant apparatus comprising:
an annular valve extending along a valve axis between an inflow end of the valve and an outflow end of the valve, the valve comprising a lumen extending along the valve axis; and
an expansion apparatus configured to engage the valve, the expansion apparatus comprising:
a shaft extending coaxially with the valve axis;
a base portion attached to the shaft;
a first arm extending between a first end and a second end, the first end pivotably attached to the base portion;
a second arm extending between a third end and a fourth end, the third end pivotably attached to the second end and the fourth end removably and pivotably attached to the valve, a central portion of the second arm pivotably attached to the shaft, wherein movement of the shaft relative to the base portion is configured to move the valve between a first position, in which the valve is in a radially-contracted position and an angle between the first arm and the second arm is greater than about 135 degrees, and a second position, in which the valve is in a radially-expanded position and the angle is less than about 135 degrees.

6. The heart valve implant apparatus of claim 5, wherein the shaft is movable along a first axis, and the second arm is configured to exert a force upon the valve along a second axis, the first axis substantially perpendicular to the second axis.

7. The heart valve implant apparatus of claim 5, wherein the valve comprises a first attachment portion positioned at a first location on an inner radial side of the valve and a second attachment portion positioned at a second location on the inner radial side, the first attachment portion spaced circumferentially apart from the second attachment portion about the valve axis.

8. The heart valve implant apparatus of claim 7, wherein the first attachment portion is configured to removably receive the first arm and the second attachment portion is configured to removably receive the second arm.

9. The heart valve implant apparatus of claim 7, wherein a distance separating the shaft from the third end and the fourth end increases as the valve moves from the first position to the second position, and wherein the valve comprises a locking frame member that resists movement of the valve from the radially-expanded position to the radially-contracted position.

10. A heart valve implant apparatus comprising:
an annular valve extending along a valve axis between an inflow end of the valve and an outflow end of the valve, the valve comprising a lumen extending along the valve axis; and
an expansion apparatus configured to engage the valve, the expansion apparatus comprising:
a shaft extending coaxially with the valve axis;
an engagement apparatus comprising:
a first base portion attached to the shaft and in a fixed position relative to the shaft;
a second base portion attached to the shaft and spaced a distance apart from the first base portion, the second base portion configured to move relative to the shaft;
an arm removably attached at one end to a first location of the valve and attached at an opposing end to the second base portion, wherein movement of the shaft is configured to change a distance between the second base portion and the first base portion such that the valve is configured to move between a first position, in which the valve is in a radially-contracted position, and a second position, in which the valve is in a radially-expanded position.

11. The heart valve implant apparatus of claim 10, wherein the second base portion is movable along a first axis, and the arm configured to exert a force upon the valve along a second axis, the first axis substantially perpendicular to the second axis.

12. The heart valve implant apparatus of claim 10, the engagement apparatus further comprising:
a third base portion attached to the shaft and in a fixed position relative to the shaft;
a fourth base portion attached to the shaft and spaced a distance apart from the third base portion, the fourth base portion configured to move relative to the shaft;
a second arm removably attached at one end to a second location of the valve and attached at an opposing end to the fourth base portion, wherein movement of the shaft is configured to change a second distance between the fourth base portion and the third base portion such that the valve is configured to move between the first position and the second position.

13. A heart valve implant apparatus comprising:
an annular valve extending along a valve axis between an inflow end of the valve and an outflow end of the valve, the valve comprising:
a first frame member comprising a first member portion comprising a first thickness and a second member portion comprising a second thickness that is less than the first thickness;
a second frame member comprising a third member portion comprising a third thickness and a fourth member portion comprising a fourth thickness that is less than the third thickness, the first frame member attached to the second frame member at an attachment location in which the second member portion is in contact with and attached to the fourth member portion such that a combined thickness of the second thickness and the fourth thickness at the attachment location is less than or equal to each of the first thickness and the third thickness; and
a locking member attached to the first frame member and configured to contact the second frame member when the valve is in a radially-expanded position to maintain the first frame member in a fixed position relative to the second frame member.

14. The heart valve implant apparatus of claim 13, wherein the locking member extends along a side of the second member portion to define an elongated opening between the locking member and the second member portion.

15. The heart valve implant apparatus of claim 14, wherein the locking member contacts the third member portion when the valve is in the radially-expanded position.
